(19) United States
(12) Patent Application Publication
(10) Pub. No.: US 2024/0032962 A1
(43) Pub. Date: Feb. 1, 2024

(54) INSERTION APPARATUS FOR AN INVASIVE PROCEDURE AND METHOD

(71) Applicant: Siemens Healthcare GmbH, Erlangen (DE)

(72) Inventor: Arne Hengerer, Möhrendorf (DE)

(21) Appl. No.: 18/220,463

(22) Filed: Jul. 11, 2023

(30) Foreign Application Priority Data
Jul. 26, 2022 (DE) 10 2022 207 649.4

(51) Int. Cl. A61B 17/34

(52) U.S. Cl. CPC A61B 17/3403; A61B 2017/00039

(57) ABSTRACT

Systems and methods for positioning and/or aligning a needle-shaped instrument more easily during an MR image-guided invasive procedure. An insertion apparatus is provided having a pen-shaped main body that has a longitudinal axis. In addition, the insertion apparatus has a guidance facility in or on the main body for guiding the predefined needle-shaped instrument parallel to the longitudinal axis of the main body. In the main body is provided a 3D magnetic field sensor for measuring magnetic field values with respect to three orthogonal spatial directions. A signal interface is used to convey the magnetic field values to an analysis facility external to the insertion apparatus.

### CROSS REFERENCE TO RELATED APPLICATIONS

This application claims the benefit of DE 10 2022 207 649.4 filed on Jul. 26, 2022, which is hereby incorporated by reference in its entirety.

### FIELD

Embodiments relate to an insertion apparatus for inserting a predefined needle-shaped instrument during an MR image-guided invasive procedure (“MR” stands for magnetic resonance).

### BACKGROUND

In minimally invasive medical procedures, medical instruments, for example catheters and/or intervention needles, are introduced into a patient, usually with image monitoring of the procedure. The image monitoring allows the acquisition of images in which the medical instrument is visualized in relation to its anatomical surroundings. While traditionally X-ray imaging has been used for image monitoring of minimally invasive medical interventions, for example fluoroscopy, it has now also been proposed to employ magnetic resonance devices, consequently magnetic resonance imaging (MR imaging), for image monitoring. This is typically referred to as interventional MR imaging. When employing what are known as closed magnetic resonance devices, that have a main magnet having a cylindrical patient placement region in which the homogeneity volume is located, work must be carried out in a really tight space and therefore any assistance to the person performing the intervention is useful.

A special type of medical instrument often used for minimally invasive medical interventions is an intervention needle, that is used, for example, for biopsy, ablation or brachytherapy. In addition, it has already been proposed with regard to intervention needles to propagate these under magnetic resonance real-time control. This requires that the intervention needle for placement is inserted at an entry position specified in a planning step and for example at a defined entry angle in order then to be fed along the thus specified trajectory to a target position, for example to a lesion.

The planning phase takes into account the anatomical circumstances and technical constraints arising from the positioning of the patient in the patient placement region. Anatomical circumstances relate for example to the localization not only of the lesion but also of bones, vessels, and other structures to be protected. The planning, for example specifying the entry position, the trajectory, and the target position, may be performed, for example, in MR images, for example three-dimensional MR images, acquired by the magnetic resonance device.

In order to perform the minimally invasive intervention in a time-efficient manner and with as much protection and as little pain for the patient as possible, it is imperative that the entry position and, if applicable, also the entry angle given by the planning may be found without subsequent repositioning.

Approaches to implementing this in a magnetic resonance device have already been proposed. In one approach, a person performing the intervention may use their finger to manually mark and represent the entry position and the entry angle. For this purpose, under image monitoring, i.e., real-time MR imaging, the radiologist's finger is positioned on the entry position in the same way as the intervention needle is meant to be inserted later. However, the large diameter of the finger compared with the diameter of the intervention needle means that accurate planning is hardly possible in this way. The entry position identified in this manner is then marked by a pen and/or by an adhesive label visible in MR image data. It is likewise known to place a grid, that is visible in MR image data, on the surface of the patient, and to determine the entry position relative to the grid, for example by counting grid lines. In another approach it has been proposed to use a laser point, or generally a light pattern, projected onto the surface of the patient to mark the location of the entry position in the longitudinal direction of the patient acquisition. Even after planning and stipulating the entry position on the basis of MR image data, these approaches allow only inexact marking. This means that the entry position used in the minimally invasive medical intervention for the medical instrument may differ from the stipulated entry position, thereby increasing the risk and duration of the minimally invasive medical intervention.

### BRIEF DESCRIPTION AND SUMMARY

The scope of the present invention is defined solely by the appended claims and is not affected to any degree by the statements within this summary. The present embodiments may obviate one or more of the drawbacks or limitations in the related art.

Embodiments provide improved assistance to a person performing an invasive medical intervention assisted by MR-imaging images.

An insertion apparatus is provided for inserting a predefined needle-shaped instrument during an MR image-guided invasive procedure. The insertion apparatus serves to assist in inserting the needle-shaped instrument into the body of a patient during this invasive procedure. The needle-shaped instrument may be an ablation needle or a biopsy needle or the like, for example. The intention then is to assist, for example a doctor, in guiding the needle-shaped instrument via a desired needle entry point to a specified needle target point, that the doctor cannot see with the naked eye. With the aid of the insertion apparatus, the needle-shaped instrument is meant to be aligned such that it may be moved linearly towards the needle target point.

For this purpose, the insertion apparatus has a pen-shaped main body having a longitudinal axis. This pen-shaped main body makes manual handling of the insertion apparatus easier, and by its longitudinal axis intuitively informs the user of the direction in which the needle-shaped instrument is to propagate. The longitudinal axis runs centrally through the main body in its longitudinal direction.

The insertion apparatus includes a guidance facility in or on the main body for guiding the predefined needle-shaped instrument parallel to the longitudinal axis of the main body. The guidance facility is configured to accommodate the needle-shaped instrument and to guide it linearly, for example parallel to the longitudinal axis of the main body. The guidance facility may be formed with the main body as a single part or in multiple parts.

In addition, the insertion apparatus includes a 3D magnetic field sensor in the main body for measuring magnetic field values with respect to three orthogonal spatial directions. The 3D magnetic field sensor is rigidly connected to the main body and hence has a fixed geometric relationship thereto. It is thus possible to deduce from the position and orientation of the 3D magnetic field sensor the position and orientation of the main body and hence also of the insertion apparatus. The 3D magnetic field sensor captures magnetic field components in all three orthogonal spatial directions. Since in an MR device the magnetic field strengths are known in all spatial directions at every point in the examination space, a position and orientation of the 3D magnetic field sensor or of the insertion apparatus may be deduced definitively from the captured magnetic field strengths or magnetic field values.

Furthermore, the insertion apparatus is equipped with a signal interface for conveying the magnetic field values to an analysis facility external to the insertion apparatus. The insertion apparatus may communicate with the external analysis facility via the signal interface. The magnetic field values may be routed via this signal interface to an analysis facility that is scaled sufficiently for the analysis and, for example, is part of the image processing facility of the MR device. The insertion apparatus is equipped to facilitate the capture of the location and orientation of the insertion apparatus in an MR device exactly.

In an embodiment of the insertion apparatus, the 3D magnetic field sensor is a 3D Hall sensor. Magnetic fields may also be measured by other sensors such as magnetometers or gradiometers. A Hall sensor has the advantage, however, that it is not only very sensitive but also robust and reliable.

In an embodiment of the insertion apparatus, the signal interface is configured for wired transfer. For example, the signal interface includes a plug-in connector, into, or onto, that may be plugged into a cable for data transfer. If applicable, a cable is also permanently connected to the signal interface. The wired transfer has the advantage especially in the MR environment of being able to keep out interference by suitable shields. It is also possible for the magnetic field values from the insertion apparatus to be transferred wirelessly to the analysis facility. In this case, suitable interference suppression measures must be provided for the wireless transfer.

In an embodiment of the insertion apparatus, the guidance facility is arranged externally on the pen-shaped main body and has a guidance axis at a displacement parallel to the longitudinal axis, and the displacement may be provided directly or indirectly via the signal interface. For example, the guidance facility is mounted as a linear guide externally on the main body. The guidance axis of the guidance facility and the longitudinal axis of the main body thus differ from each other and include a certain displacement or offset. In order that the needle-shaped instrument may be guided on a defined needle trajectory, it is therefore necessary to know the displacement between the longitudinal axis and the guidance axis if the insertion apparatus but not the needle-shaped instrument itself is visible in the MR image. Then, by the known displacement, the insertion apparatus may be placed and/or aligned such that the needle-shaped instrument may also actually be inserted on a planned needle trajectory. It is necessary here to provide the displacement between both axes to the analysis facility. For example, this displacement may be transferred directly as a specific value from the signal interface to the analysis facility. If applicable, however, the displacement may also be provided indirectly via specific coding. For example, a connector coding at the signal interface could be used to transfer the displacement in coded form to the analysis facility.

The guidance facility may also be arranged centrally in the main body and include a guidance axis that is identical to the longitudinal axis of the main body. In this case, similar to a ballpoint pen, in which the refill runs axially in the center, the guidance facility may also guide the needle-shaped instrument centrally on the longitudinal axis of the main body. In a simple case, the main body has for this purpose a central hole as the guidance facility, through which the needle-shaped instrument may be guided. This central guidance in the main body has the advantage that there is no offset between the guidance axis and the longitudinal axis of the main body, and hence the longitudinal axis of the main body automatically specifies the position of the needle-shaped instrument.

In certain embodiments, the needle-shaped instrument has respectively a catheter, a guidewire, a brachytherapy needle, a biopsy needle or an ablation needle. Embodiments are not limited to the aforementioned needle-shaped instruments. The insertion apparatus has the advantage, however, that these instruments may reach a desired location in the patient in a targeted manner without repositioning.

A magnetic resonance system is also provided including a magnet unit for producing a magnetic field in an examination space; an MR image processing facility for producing an MR image according to the magnetic field in the examination space; an insertion apparatus as claimed in one of the preceding claims; and a signal processing facility for capturing magnetic field values conveyed from the insertion apparatus, and for producing image signals from the magnetic field values for representing a location and/or an alignment of the insertion apparatus in the MR image of the MR image processing facility.

The magnetic resonance system may be a standard magnetic resonance tomography apparatus (MRT apparatus). An object to be examined (for example patient or part of a patient) is placed in an examination space of the magnet unit producing the magnetic field. The MR image processing facility produces from the detected magnetic field signals an MR image of the object in the examination space. The magnetic resonance system includes an insertion apparatus of the aforementioned type. The insertion apparatus is used to facilitate an image-guided invasive procedure that may be performed with greater safety. The signal processing facility of the MR system is used here not only for capturing the magnetic field values conveyed from the insertion apparatus but also to produce a corresponding image of the insertion apparatus in the MR image. This is done, for example, by transferring the image signals relating to the insertion apparatus to the MR image processing facility so that it may produce a combined image of the examination space and of the insertion apparatus.

A method is provided for placing and aligning a needle-shaped instrument for an MR image-guided invasive procedure, the method including specifying a needle trajectory for the needle-shaped instrument, placing an insertion apparatus, that has the needle-shaped instrument and a 3D magnetic field sensor, in an examination space, producing an MR image of the examination space with a representation of the needle trajectory, automatically capturing signals from the 3D magnetic field sensor of the insertion apparatus in the examination space, and depicting the insertion apparatus including the needle-shaped instrument accurately in terms of position and orientation in the MR image on the basis of the captured signals from the 3D magnetic field sensor.

The advantages and developments outlined above in connection with the insertion apparatus and the magnetic resonance system apply mutatis mutandis also to the method. Accordingly, the presented functional features of the apparatus or of the system may be regarded in the case of the method as corresponding method features.

A computer program is also provided that includes commands that, on execution of the program by an aforementioned apparatus, cause the apparatus to execute the method also described above. In addition, a computer-readable storage medium may be provided that includes commands that, on execution by the above apparatus, cause the apparatus to execute the aforementioned method. For example, the storage medium may be configured at least in part as a non-volatile data storage device (for example as a flash memory and/or as an SSD—solid state drive) and/or at least in part as a volatile data storage device (for example as a RAM—random access memory). In addition, the storage medium may be realized in a data storage device of a processor circuit. However, the storage medium may also be operated in the Internet as what is known as an App Store Server, for example. A processor circuit including at least one microprocessor may be provided by a computer or computer network. The commands may be provided as binary code or assembler and/or as source code of a programming language (for example C).

For cases of use and situations of use that may arise in the method and are not explicitly described here, it may be provided that, according to the method, an error message and/or a prompt to input user feedback is output and/or a default setting and/or a predetermined initial state is set.

### BRIEF DESCRIPTION OF THE FIGURES

FIG. 1 depicts a schematic view of a magnetic resonance system according to an embodiment.

FIG. 2 depicts a perspective view of an embodiment of an insertion apparatus.

FIG. 3 depicts an example of an MR image with needle trajectory and insertion apparatus according to an embodiment.

FIG. 4 depicts a further MR image with needle trajectory and depicted insertion apparatus according to an embodiment.

FIG. 5 depicts a flow diagram of an embodiment of a method.

### DETAILED DESCRIPTION

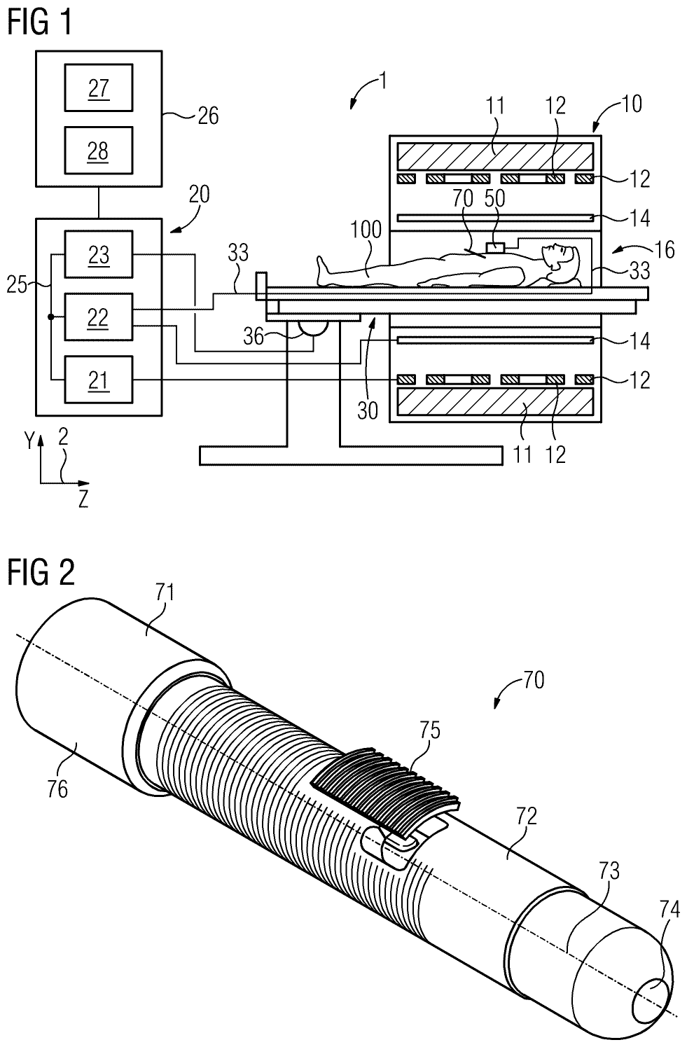

FIG. 1 depicts a schematic diagram of a magnetic resonance tomography unit, i.e., of a magnetic resonance system 1, for use with the insertion apparatus.

The magnet unit 10 includes a field magnet 11, that produces a static magnetic field B0 for aligning nuclear spins of samples or of the patient 100 in an acquisition region. The acquisition region is characterized by an extremely homogeneous static magnetic field B0, the homogeneity relating for example to the magnetic field strength or magnitude. The acquisition region is approximately spherical and located in a patient tunnel 16, that extends through the magnet unit 10 in a longitudinal direction 2.

A patient couch 30 may be moved inside the patient tunnel 16 by the travel unit 36.

The field magnet 11 is usually a superconducting magnet, that may provide magnetic fields having a magnetic flux density of up to 3T or even higher in the latest equipment. For lower field strengths, however, permanent magnets or electromagnets having normal-conducting coils may also be used.

The magnet unit 10 also has gradient coils 12, that are configured to superimpose variable magnetic fields in three spatial dimensions on the magnetic field B0 for the purpose of spatial discrimination of the acquired imaging regions in the examination volume. The gradient coils 12 are usually coils made of normal-conducting wires, that may generate mutually orthogonal fields in the examination volume.

The magnet unit 10 also has a body coil 14, that is configured to radiate into the examination volume a radiofrequency signal supplied via a signal line, and to receive resonance signals emitted by the patient 100 and to output the resonance signals via a signal line. The term transmit antenna denotes below an antenna via which is emitted the radiofrequency signal for exciting the nuclear spins. This may be the body coil 14 but may also be a local coil 50 having a transmit function.

A control unit 20 supplies the magnet unit 10 with the various signals for the gradient coils 12 and the body coil 14 and analyzes the received signals.

The control unit 20 includes a gradient controller 21, that is configured to supply the gradient coils 12 via supply lines with variable currents that provide, coordinated in time, the desired gradient fields in the examination volume.

In addition, the control unit 20 includes a radiofrequency unit 22, that is configured to produce a radiofrequency pulse having a predefined variation over time, amplitude and spectral power distribution for the purpose of exciting magnetic resonance of the nuclear spins in the patient 100. Pulse powers may reach in the region of kilowatts here. The excitation signals may be radiated via the body coil 14 or via a local transmit antenna into the patient 100.

A controller 23 communicates via a signal bus 25 with the gradient controller 21 and the radiofrequency unit 22.

Arranged on the patient 100 is a local coil 50, that is connected via a connecting line 33 to the radiofrequency unit 22 and its receiver. It is also conceivable, however, that the body coil 14 is a receive antenna.

By an insertion apparatus 70, a needle-shaped instrument, for example a biopsy needle or an ablation needle, may be inserted into the patient 100 in a targeted manner. FIG. 1 depicts the insertion apparatus 70 together with the needle-shaped instrument as a thick line.

The insertion apparatus 70 includes a magnetic field sensor, for example a Hall sensor 71 (cf. FIG. 2). This magnetic field sensor is configured for 3D magnetic field measurement. Hence the magnetic field sensor may determine in the patient tunnel 16, according to its position and alignment, magnetic field values for three spatial directions.

The Hall sensor 71 or the magnetic field sensor is in communication connection with a signal processing facility 28, that may be part of an image processing facility 26 of the magnetic resonance system 1 or of the control unit 20. The communication connection is not shown in FIG. 1 for the sake of clarity and may be wired or wireless.

The image processing facility 26 may also include a display facility 27 for reproducing one or more MR images from the magnetic resonance system 1.

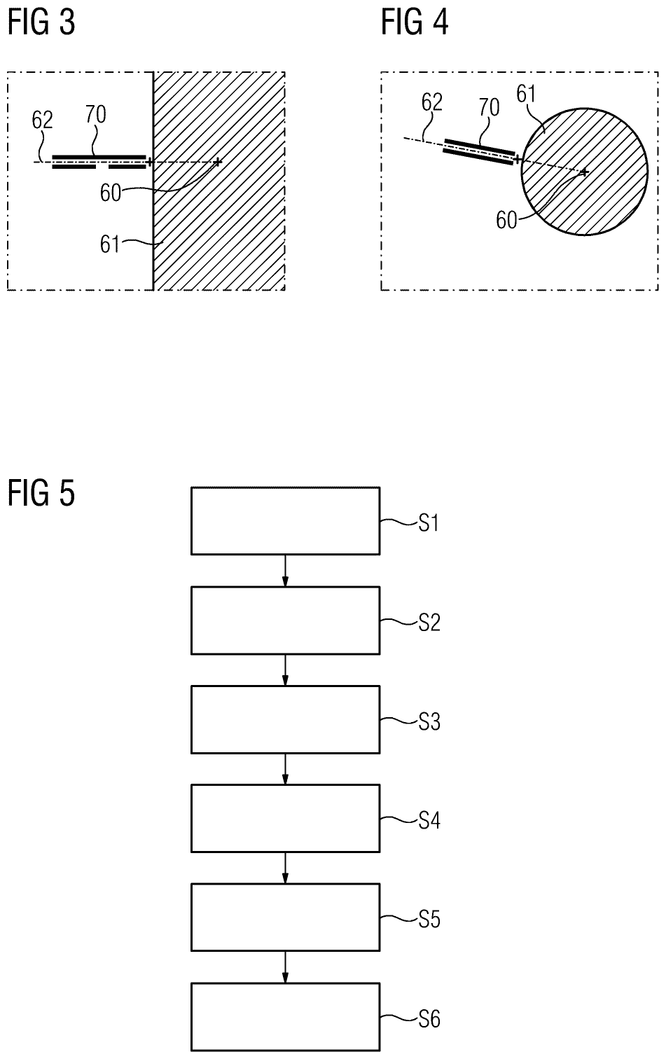

The signal processing facility 28 is also capable of producing from the magnetic field values measured by the Hall sensor 71 image signals relating to the insertion apparatus 70. The image processing facility 26 may combine these image signals with the standard MR image from the patient tunnel 16 so that in the MR image the insertion apparatus 70 may be seen in its position and/or alignment. Either the MR image is a three-dimensional representation, so that both the position and the orientation of the insertion apparatus 70 are easily identifiable for the operator, or, for example, it is one or more 2D acquisitions as shown in FIG. 3 and FIG. 4, that show the object in the patient tunnel including the insertion apparatus from one or more perspectives.

FIG. 2 depicts an example of a pen-shaped insertion apparatus 70. It has a main body 72 that is likewise pen-shaped. The main body 72 has a longitudinal axis 73. This longitudinal axis 73 here also forms the center axis of the hollow-cylindrical main body 72.

The insertion apparatus 70 includes a tubular guidance facility 74 along the longitudinal axis 73. This tubular guidance facility may extend through the entire length of the insertion apparatus or else just through a portion thereof. In this guidance facility 74 is guided the needle-shaped instrument (not shown here) along a guidance axis. In the present example, this guidance axis corresponds to the longitudinal axis 73. Alternatively, however, the insertion apparatus may also include externally on the pen-shaped main body a guidance facility for guiding the needle-shaped instrument. Again then, the guidance axis would be parallel to the longitudinal axis 73, but there would be a certain offset between both axes that must be taken into account in the representation of the insertion apparatus 70 in the MR image.

If applicable, the insertion apparatus 70 includes a slider 75 for moving the needle-shaped instrument, that is located in the guidance facility 74, along the guidance axis. Also, other sliding or movement options (if applicable also of an automatic nature) are conceivable on the insertion apparatus 70.

The insertion apparatus 70 also includes a 3D magnetic field sensor, in the present example the Hall sensor 71. It may be used to ascertain precise magnetic field values in three orthogonal spatial directions. Since the precise magnetic field distribution in the patient tunnel 16 is known, this may also be used to determine an exact position and orientation of the insertion apparatus 70 in the examination space or the patient tunnel 16 of the magnetic resonance system 1.

The “Hall pen” shown in FIG. 2 thus facilitates by its vertical and horizontal sensor elements a 3D magnetic field measurement in the patient tunnel 16. Since, as already suggested, the “field map” (magnetic field distribution) of any MRT device may be determined, it is hence possible by the Hall effect to localize the angular rotation and 3D position of the “Hall pen” (i.e., of the insertion apparatus 70) definitively in the patient tunnel 16 (if applicable taking into account the offset between longitudinal axis 73 and guidance axis). This may make use of the fact that the output voltage of the respective Hall sensors depends on the strength and the direction of the magnetic field in the patient tunnel 16.

The “Hall pen” may be wired. The energy supply and the signal communication are made via the cable. A wireless version of the “Hall pen” having rechargeable battery and, for example, Bluetooth connection, is also conceivable, however, even though this is technically more difficult to realize because of the MR magnetic field.

3D Hall sensors are available at low cost and in a very compact design (for example 25×35 mm) If applicable, the “Hall pen” may even be produced as a sterile disposable product.

FIGS. 3 and 4 show a practical implementation of the method for placing and aligning a needle-shaped instrument for an MR image-guided invasive procedure. In the present example, the needle-shaped instrument (for example biopsy needle) is meant to be placed at a target point 60 of a cylindrical phantom 61. FIG. 3 depicts the cylindrical phantom 61 from the side, and FIG. 4 depicts it in a plan view. In both cases, the image is an MRT image or CT image, for example.

A planned needle trajectory 62 is overlaid in the respective images.

In addition, a double line representing an insertion apparatus 70 may be seen on the MR or CT image of both FIG. 3 and FIG. 4. As one alternative, the depiction of the insertion apparatus 70 is obtained automatically during the imaging, for example because the material of the insertion apparatus exhibits an appropriate magnetic interaction. In the present case, however, the magnetic field sensor of the insertion apparatus 70 captures its position and orientation so that a graphic representing the insertion apparatus may be overlaid in the respective images accurately in terms of position and orientation.

In the present example, a guidance channel for guiding the needle-shaped instrument is evident in the center of the respective double lines representing the insertion apparatus 70. The needle-shaped instrument therefore runs exactly between the double line for the invasive procedure. The doctor performing the treatment may now align the insertion apparatus 70 both in the side view and in the plan view such that the center of the double line lies on the planned trajectory 62. It is hence guaranteed that when the needle-shaped instrument is being inserted by the insertion apparatus 70, the needle-shaped instrument actually reaches the needle target point 60, that is not visible externally.

In an embodiment, that is not illustrated, the insertion apparatus 70, that is pen-shaped, is visible or represented in the image only by a single line, if applicable. In this case, if applicable, only this single line must then be aligned on the planned needle trajectory 62. If applicable, the illustration of the needle trajectory 62 or of the insertion apparatus 70 takes into account an offset that exists between the longitudinal axis 73 and a guidance axis on which the needle-shaped instrument is guided longitudinally.

To perform the intervention using the needle-shaped instrument, intervention software may be provided, that, for example, runs on an MRT host and has the following functionalities: planning of the needle trajectory 62 by marking the needle entry point and the needle target point 60, if applicable taking into account the offset of the Hall pen and the needle; automatic detection of the Hall pen, i.e. of the insertion apparatus 70; extracorporeal projection of the needle trajectory (extrapolation of the needle trajectory outwards via the needle entry point) and visual or acoustic feedback when the user has positioned the Hall pen correctly on the needle entry point; further visual or acoustic signal when the Hall pen is aligned correctly in accordance with the extracorporeal projection of the needle trajectory.

The sequence of an example of a method is explained in greater detail in connection with FIG. 5. In a step S1, a needle trajectory 62 for the needle-shaped instrument is specified. If applicable, this is done by specifying a needle entry point and a needle target point 60. A straight line between both points gives the needle trajectory in the patient or phantom 61. If applicable, the needle trajectory may be extrapolated outside the needle entry aperture.

In a further step S2, the insertion apparatus 70, that has the needle-shaped instrument and a 3D magnetic field sensor, is placed in the examination space, for example patient tunnel 16. The insertion apparatus 70 is thereby present in the examination space but is not yet aligned and is not yet positioned at the desired location.

In a further step S3, an MR image of the examination space with a representation of the needle trajectory 62 is produced. The virtual needle trajectory is thus overlaid in the MR image.

In a further step S4, signals from the 3D magnetic field sensor 71 of the insertion apparatus 70 are captured automatically in the examination space. For example, the automatic capture is performed by a signal processing facility 28 of an image processing facility 26 of the magnetic resonance system 1.

In a further step S5, the insertion apparatus 70, if applicable including the needle-shaped instrument, is depicted accurately in terms of position and orientation in the MR image on the basis of the captured signals from the 3D magnetic field sensor. Thus, in the MR image appears not only the specified needle trajectory 62 but also a depiction or overlay of a (synthetic) image of the insertion apparatus 70. This image of the insertion apparatus 70 is accurate not only in terms of position but also in terms of orientation in relation to the actual pose of the insertion apparatus 70 in the examination space. This means that the insertion apparatus 70 is represented in the MR image as it would actually be depicted in the MR image by the MR scanner. In fact, however, a synthetic image of the insertion apparatus 70 is represented accurately at the image position that corresponds to the actual position. Equally, the image orientation of the insertion apparatus 70 is represented exactly as the actual orientation of the insertion apparatus 70. Since the insertion apparatus 70 may move freely in space, it is possible from the MR image (if applicable also from a plurality of MR images) to place the insertion apparatus on the specified needle trajectory 62 and align it there.

For example, the insertion apparatus is placed with the tip of the needle-shaped instrument at the planned needle entry aperture. The specified needle trajectory 62 may also be used to align the entire insertion apparatus so as to adopt the desired spatial angle. This placement and alignment of the insertion apparatus 70 constitutes a further step S6 in the flow diagram of FIG. 5. The placement and alignment may be performed manually or else by robot or automatically. For example, the placement and alignment of the insertion apparatus may be made on the needle trajectory 62 or, if applicable, also beside the needle trajectory at a predetermined displacement.

Rapid and intuitive needle positioning is possible using the insertion apparatus presented above and using the corresponding method. The Hall pen presented may be more cost-effective than optical navigation systems derived from neurosurgical methods. In addition, the Hall pen is user friendly in the spatially confined MRT patient tunnel, because the user does not need to take care to avoid shadowing the navigation system.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. An insertion apparatus for inserting a predefined needle-shaped instrument during an MR image-guided invasive procedure, the insertion apparatus comprising:
a pen shaped main body including a longitudinal axis;
a guidance facility in or on the pen shaped main body, the guidance facility configured for guiding the predefined needle-shaped instrument parallel to the longitudinal axis of the pen shaped main body;
a 3D magnetic field sensor in the pen shaped main body, the 3D magnetic field sensor configured for measuring magnetic field values with respect to three orthogonal spatial directions; and
a signal interface configured for conveying the magnetic field values to an analysis facility external to the insertion apparatus.

2. The insertion apparatus of claim 1, wherein the 3D magnetic field sensor is a 3D Hall sensor.

3. The insertion apparatus of claim 1, wherein the signal interface is configured for wired transfer.

4. The insertion apparatus of claim 1, wherein the guidance facility is arranged externally on the pen shaped main body and includes a guidance axis at a displacement parallel to the longitudinal axis, wherein the displacement may be provided directly or indirectly via the signal interface.

5. The insertion apparatus of claim 1, wherein the guidance facility is arranged centrally in the main body and has a guidance axis that is identical to the longitudinal axis of the main body.

6. The insertion apparatus of claim 1, wherein the predefined needle-shaped instrument includes a catheter, a guidewire, a brachytherapy needle, a biopsy needle or an ablation needle.

7. A magnetic resonance system comprising:
a magnet unit configured to produce a magnetic field in an examination space;
an MR image processing facility configured to produce an MR image according to the magnetic field in the examination space;
an insertion apparatus comprising:
a pen shaped main body including a longitudinal axis;
a guidance facility in or on the pen shaped main body, the guidance facility configured for guiding a needle shaped instrument parallel to the longitudinal axis of the pen shaped main body;
a 3D magnetic field sensor in the pen shaped main body, the 3D magnetic field sensor configured for measuring magnetic field values with respect to three orthogonal spatial directions;
a signal interface configured for conveying the magnetic field values to an analysis facility external to the insertion apparatus; and
a signal processing facility configured to capture magnetic field values conveyed from the insertion apparatus and to produce image signals from the magnetic field values for representing a location, an alignment, or the location and the alignment of the insertion apparatus in the MR image of the MR image processing facility.

8. The magnetic resonance system of claim 7, wherein the 3D magnetic field sensor is a 3D Hall sensor.

9. The magnetic resonance system of claim 7, wherein the signal interface is configured for wired transfer.

10. The magnetic resonance system of claim 7, wherein the guidance facility is arranged externally on the pen shaped main body and includes a guidance axis at a displacement parallel to the longitudinal axis, wherein the displacement may be provided directly or indirectly via the signal interface.

11. The magnetic resonance system of claim 7, wherein the guidance facility is arranged centrally in the main body and has a guidance axis that is identical to the longitudinal axis of the main body.

12. The magnetic resonance system of claim 7, wherein the needle shaped instrument includes a catheter, a guidewire, a brachytherapy needle, a biopsy needle or an ablation needle.

13. A method for placing and aligning a needle-shaped instrument for an MR image-guided invasive procedure, the method comprising:
specifying a needle trajectory for the needle-shaped instrument;
placing an insertion apparatus that includes the needle-shaped instrument and a 3D magnetic field sensor, in an examination space;
producing an MR image of the examination space with a representation of the needle trajectory;
automatically capturing signals from the 3D magnetic field sensor of the insertion apparatus in the examination space; and
depicting the insertion apparatus including the needle-shaped instrument accurately in terms of position and orientation in the MR image on a basis of the captured signals from the 3D magnetic field sensor.

14. The method of claim 13, wherein the needle trajectory in the MR image is specified by marking a needle entry point and a needle target point.

15. The method of claim 14, wherein the needle trajectory is extrapolated beyond the needle entry point by an extrapolation trajectory.

16. The method of claim 15, wherein the insertion apparatus is positioned and aligned in the examination space by the extrapolation trajectory of the MR image.

17. The method of claim 13, wherein the 3D magnetic field sensor is a 3D Hall sensor.

18. The method of claim 13, wherein the needle-shaped instrument includes a catheter, a guidewire, a brachytherapy needle, a biopsy needle or an ablation needle.
